(19) United States
(12) Patent Application Publication
(10) Pub. No.: US 2025/0127716 A1
(43) Pub. Date: Apr. 24, 2025

(54) INHALED IMATINIB FOR TREATMENT OF PULMONARY HYPERTENSION

(71) Applicant: JUSTUS-LIEBIG-UNIVERSITAET GIESSEN, Giessen (DE)

(72) Inventors: Werner Seeger, Biebertal (DE); Tobias Gessler, Wettenberg (DE); Alexandra C. Dalla-Bona, Geissen (DE)

(21) Appl. No.: 18/835,675

(22) PCT Filed: Feb. 4, 2022
(86) PCT No.: PCT/EP2022/052664

§ 371 Date: Aug. 2, 2024

(51) Int. Cl. A61K 9/00; A61K 9/08; A61K 31/506; A61K 47/10

(52) U.S. Cl. CPC A61K 9/0078; A61K 9/08; A61K 31/506; A61K 47/10

(57) ABSTRACT

Provided herein are methods for treating pulmonary hypertension. The methods include formulation of stable and highly concentrated imatinib solutions, selection of inhalers and nebulizers capable of aerosolizing such stable and highly concentrated imatinib solutions and administering to a subject an effective dose of imatinib, wherein imatinib solution is aerosolized using a nebulizer and inhaled by the subject. In preferred embodiments, the nebulizer is selected from the group of soft mist inhalers, such as the Medspray™ wet aerosol inhaler or the Respimat™.

A method of treating a patient suffering from pulmonary hypertension, comprising:
(a) providing stable and highly concentrated imatinib solutions
(b) providing an aerosol producing device capable of aerosolizing such stable and highly concentrated imatinib solutions
(b) administering to the patient an effective dose of imatinib by inhalation.

### FIELD OF INVENTION

The present invention relates to methods, compositions and kits for the treatment of patients with pulmonary hypertension by imatinib aerosol therapy.

### BACKGROUND OF THE INVENTION

Imatinib is a 2-phenyl amino pyrimidine derivative used as oral chemotherapy medication to treat different types of cancer, e.g. chronic myelogenous leukemia, acute lymphocytic leukemia, gastrointestinal stromal tumors, hypereosinophilic syndrome, chronic eosinophilic leukemia, systemic mastocytosis, and myelodysplastic syndrome.

According to IUPAC the chemical name of imatinib is 4-[(4-methylpiperazin-1-yl)methyl]-N-(4-methyl-3-{[4-(pyridin-3-yl)pyrimidin-2-yl]amino}phenyl)benzamide. Imatinib acts as a specific inhibitor of a number of tyrosine kinase enzymes occupying their ATP-binding site and thereby leading to a decrease in activity. Imatinib is available as film-coated tablets containing imatinib mesylate for oral therapy.

Pulmonary hypertension (PH) is a severe and potentially life-threatening disease defined by an increase in mean pulmonary arterial pressure above 25 mmHg. Common signs and symptoms of pulmonary hypertension include shortness of breath (dyspnea), exercise intolerance, fatigue, dizziness or syncope, chest pressure or pain, edema formation, cyanosis, tachycardia and heart palpitations. Pulmonary hypertension is currently classified by the WHO into the following five groups: Group 1, Pulmonary arterial hypertension (PAH); Group 2, Pulmonary hypertension due to left heart disease; Group 3, Pulmonary hypertension due to lung disease and/or hypoxia; Group 4, Pulmonary hypertension due to pulmonary artery obstructions; Group 5, Pulmonary hypertension with unclear and/or multifactorial mechanisms. Deciphering the pathophysiological background of PH has facilitated the development of specific PH medication over the last decades, especially for group 1 and 4 PH.

Currently, there are several PH-specific medications available, addressing the three principal signaling pathways of pulmonary vasoregulation: the prostacyclin-pathway with epoprostenol (intravenous), iloprost (inhaled, intravenous), treprostinil (inhaled, intravenous, subcutaneous, oral), beraprost (oral) and selexipag (oral); the nitric oxide-pathway with sildenafil (oral), tadalafil (oral), vardenafil (oral) and riociguat (oral); the endothelin-pathway with bosentan (oral), ambrisentan (oral) and macitentan (oral). These mainly vasodilatory drugs have considerably improved therapy of P(A)H including amelioration of clinical symptoms, deceleration of disease progression and prolonged survival. Despite this progress, however, there is still no cure for this disease; disease progression is mostly inevitable and mortality remains unacceptably high. Recently, new scientific evidence supports the concept of PH as disease with “cancer-like” hyperproliferative nature, with sustained proliferative signaling, evasion of growth suppressors, resistance to apoptosis, deregulation of cellular energetics, limit-less replicative potential and DNA instability due to epigenetic and genetic alterations, activation of specific signal transduction, chronic inflammation, pathological angiogenesis, and immune system evasion. Thus, identification of new molecular strategies targeting the remodeling of diseased pulmonary arteries and introduction of new game-changing drugs based on this concept are urgently needed.

Imatinib was the first anti-proliferative drug investigated in PAH (Ghofrani et al., N Engl J Med. 2005 353(13):1412-3). To assess safety, tolerability and efficacy of oral imatinib mesylate in PAH, a phase II pilot study with 59 patients was performed. While not meeting the primary endpoint defined as change from baseline in the 6-minute-walk distance, pulmonary hemodynamics as secondary endpoint significantly improved in the imatinib group, tolerability in the small number of patients was acceptable (Ghofrani et al., Am J Respir Crit Care Med. 2010 182(9):1171-7). In a randomized, double-blind, placebo-controlled 24-week trial in 202 PAH-patients, oral imatinib mesylate significantly improved 6-minute walk distance as primary outcome as well as pulmonary vascular resistance (Hoeper et al., Circulation 2013 127(10):1128-38). The severe adverse events, significant side effects, and a high discontinuation rate in the study and in the following open-label extension phase, however, limit the utility of oral imatinib; off-label use of this compound in PAH is discouraged (Frost et al., J Heart Lung Transplant 2015 34(11):1366-75).

### Problem

The failed development of imatinib for treatment of PAH can mainly be attributed to the route of administration of this highly effective drug. The oral application of effective doses of imatinib resulted in severe systemic adverse events limiting its further use. A formulation for imatinib is being sought that overcomes the disadvantages of oral administration and enables improved treatment of PAH.

### Solution

To solve this problem, a formulation of imatinib suitable for inhaled administration is provided according to the claims.

Provided herein are new methods for treating pulmonary hypertension. The methods include formulation of stable and highly concentrated imatinib solutions, selection of inhalers and nebulizers capable of aerosolizing such stable and highly concentrated imatinib solutions and administering to a subject an effective dose of imatinib, wherein imatinib solution is aerosolized using a nebulizer and inhaled by the subject. In preferred embodiments, the nebulizer is selected from the group of soft mist inhalers, such as the Medspray™ wet aerosol inhaler or the Respimat™.

Topical administration of drugs via the inhalative route, provides pulmonary selectivity necessitating drastically reduced doses for the desired therapeutic effects. This concept has been validated by the successful development of the vasodilatory prostacyclin analogues iloprost and treprostinil for PH.

In the phase II and phase III trial patients received 200 mg oral imatinib mesylate once daily, the dose was increased to 400 mg once daily if the starting dose of 200 mg was tolerated. The larger favorable treatment effects in the phase III study were observed in patients who received a dose of 400 mg per day with not differences in adverse events compared to the lower dose. Therefore, a target dose of 400 mg oral imatinib mesylate seems to represent the optimal and required dose to treat pulmonary hypertension, although the minimum efficacious dose of imatinib in PH has not yet been evaluated in clinical trials. Oral imatinib mesylate demonstrates almost complete bioavailability indicative of high permeability and a low hepatic extraction (Peng et al., J Clin Pharmacol. 2004; 44(2):158-62).

The inter-subject variation in pharmacokinetics of imatinib mesylate is high, presumably due to variability in the activity of cytochrome P450 isoenzyme 3A4 between individuals.

In consideration of the dose-saving aspect of an inhalative approach, a significant lower inhaled dose of approximately one-half to one-twentieth of the oral 400 mg imatinib can be projected to achieve the full efficacy profile of the drug, thereby minimizing systemic side effects.

To deliver doses of approximately 10 mg to 250 mg imatinib by inhalation, strategies for efficient nebulization of imatinib are required to avoid lengthy inhalation maneuvers. Such efficient strategies firstly depend on imatinib formulations and compositions suitable for nebulization. Secondly, the choice of the nebulizer system is crucial to achieve high drug output and therefore short inhalation times. Is it an aspect of the present invention to provide formulations and compositions with high imatinib concentration suitable for nebulization by soft mist inhalers or modern nebulizers.

In summary, it is an object of the present invention to provide methods, compositions and kits or administering imatinib as aerosol therapy to treat pulmonary hypertension, which overcome at least one of the disadvantages and shortcomings known from oral or other inhaled imatinib therapies.

### SUMMARY OF THE INVENTION

Provided herein are methods for treating pulmonary hypertension, including formulation of stable and highly concentrated imatinib solutions, selection of inhalers and nebulizers capable of aerosolizing such stable and highly concentrated imatinib solutions and administering to a subject an effective dose of imatinib by inhalation. The optimized stable and highly concentrated imatinib solutions are aerosolized using a nebulizer and inhaled by the subject. In preferred embodiments, the nebulizer is selected from the group of soft mist inhalers, such as the Medspray™ wet aerosol inhaler or the Respimat™.

Imatinib is a tyrosine kinase inhibitor approved as oral chemotherapy containing imatinib mesylate to treat different forms of cancer. Due to its anti-proliferative properties, imatinib was successfully investigated in pulmonary hypertension, a disease of the pulmonary arteries characterized by cancer-like remodeling.

Severe systemic adverse events following oral administration, however, prevented the clinical use of oral imatinib mesylate in pulmonary hypertension. Topical administration of imatinib via the inhalative route facilitates pulmonary selectivity resulting in markedly reduced doses for the desired therapeutic effects.

The invention provides formulation and composition of stable and highly concentrated liquid solutions of imatinib salts, employment of inhalers and nebulizers capable of aerosolizing such stable and highly concentrated imatinib solutions and administering to a subject an effective dose of imatinib, wherein imatinib solution is aerosolized using a nebulizer and inhaled by the subject. The subject suffers from pulmonary hypertension, and is either therapy-naïve or receiving supportive therapy and/or approved PH-specific drugs, alone or in combination.

Further aspects and embodiments will become clear on the basis of the detailed description below, the examples, and the patent claims.

### DETAILED DESCRIPTION OF THE INVENTION

The invention provides methods, compositions and kits for administering imatinib as aerosol therapy to treat pulmonary hypertension. The invention includes formulation of stable and highly concentrated imatinib solutions designed for optimal nebulization, selection of suitable inhalers or nebulizers for inhalative delivery of such highly concentrated imatinib solutions and the use of the drug-device combinations for aerosol therapy of pulmonary hypertension.

### 1) Stable and Highly Concentrated Imatinib Solutions

To deliver efficacious doses of inhaled imatinib by soft mist inhalers or modern nebulizers within short periods of time, highly-concentrated and stable liquid solutions of imatinib have to be formulated. In order to provide such imatinib solutions, imatinib free base and different salts of imatinib were dissolved in different solvents.

### a) Imatinib Free Base

Imatinib free base is poorly soluble in water, maximum solubility in pure water is estimated to be only about 5-20 μM. In order to improve solubility of imatinib free base, different water-based solvents were tested (example 1).

### Example 1

Imatinib free base (AbMole BioScience Inc.) was weighted by an analytical balance and diluted with different solvents in a 2 ml Eppendorf cup. The resulting solution was vortexed for several minutes and stored at room temperature for one hour. The following solvents were tested: aqua destillata (a. dest), physiologic saline (NaCl 0.9%), citrate buffer (5% v/v), urea 5% (v/v) in a. dest, ethanol (10% v/v) and glycerol (1% v/v) in a. dest.

Regardless of the used solvent, it was impossible to formulate stable imatinib free base solutions with an imatinib concentration significantly higher than 5 mg/ml.

### b) Imatinib Hydrochloride

Imatinib hydrochloride is also poorly soluble in water. To improve solubility of imatinib hydrochloride different water-based solvents were tested (example 2).

### Example 2

Imatinib hydrochloride (ApexBio Technology) was weighted by an analytical balance and diluted with different solvents in a 2 ml Eppendorf cup. The resulting solution was vortexed for several minutes and stored at room temperature for one hour. The following solvents were tested: aqua destillata (a. dest), physiologic saline (NaCl 0.9%), citrate buffer (5% v/v), urea 5% (v/v) in a. dest, ethanol solution (10% v/v) and glycerol (1% v/v) in a. dest.

Regardless of the used solvents, it was impossible to formulate stable imatinib hydrochloride solutions with an imatinib concentration significantly higher than 5 mg/ml.

### c) Imatinib Mesylate

Imatinib mesylate is known to display a fairly high solubility in water. With regard to the inhalative delivery in humans, different water-based solvents were selected and tested regarding the maximum imatinib concentration and stability of the solutions (example 3).

### Example 3

Imatinib mesylate (MedChemExpress LLC) was weighted by an analytical balance and diluted with different solvents in a 2 ml Eppendorf cup. The resulting solution was vortexed for several minutes and stored at room temperature for one hour. The following solvents were tested: physiologic saline (NaCl 0.9%), HCl/NaOH (<1% v/v) in a. dest with different pH, citrate buffer (1% to 5% v/v), HEPES buffer (1 mM), trometamol buffer (1 mM), PBS buffer (1 mM).

By means of these water-based solvents, it was impossible to formulate stable imatinib mesylate solutions with concentrations of 50 mg/ml or more. In general, the resulting solutions were not stable, precipitation of imatinib crystals was observed.

In summary, solvents of higher ionic strength are generally not suitable for the preparation of stable and highly concentrated imatinib mesylate solutions. Therefore, solvents of low ionic strength were used to prepare highly-concentrated solutions of imatinib mesylate (example 4).

### Example 4

Imatinib mesylate (MedChemExpress LLC) was weighted by an analytical balance and diluted with different solvents in a 2 ml Eppendorf cup. The resulting solution was vortexed for several minutes and stored at room temperature for one hour. The following solvents were tested: a. dest, ethanol 96% (v/v), ethanol in a. dest (up to 96% v/v), urea (up to 7.5% v/v), glycerol in a. dest (up to 20% v/v), mannitol in a. dest (up to 20% v/v), propylene glycol (up to 90% v/v), ethylene glycol (up to 90% v/v), polyethylene glycol (up to 90% v/v), ectoin (up to 20% v/v), and ethanol in a. dest (1, 5 or 10% v/v)+glycerol (1% v/v).

For all tested solvents, it was possible to formulate stable solutions of imatinib mesylate with concentrations up to 500 mg/ml. Regarding the lyotropic properties of imatinib, the addition of chaotropic compounds such as ethanol, urea, aldols, propylene glycol, ethylene glycol, polyethylene glycol and ectoin enhances the solubility and stability of water-based imatinib solutions. The use of such compounds in variable concentration, either alone or in composition, as solvents for imatinib mesylate is claimed in the present invention.

### d) Imatinib Addition Salts

Besides imatinib mesylate numerous imatinib addition salts have been described with specific solubility in water-based solvents. It is within the scope of the present invention to use imatinib addition salts for the preparation of suitable imatinib formulations for inhalative delivery by soft mist inhalers or modern nebulizers. Therefore, imatinib addition salts like tartrate, citrate, maleate, fumarate, succinate, benzoate, besylate, tosylate, palmoate, formate, malonate, napsylate, salysilate, cyclohexane sulfamate, lactate, mandelate, glutarate, adipate, squarate, vallinate, oxaloacetate, ascorbate and sulfate salts, oxalate, p-toluene sulfonate, naphthalene sulfonate, benzene sulfonate, nitrate, phosphate, acetate, lysinate, lysinate-HCL or arginate are included in the present invention. Preferably, the imatinib addition salt is selected from the group of highly water-soluble salts including maleate, tartrate, malonate, succinate, tosylate, oxalate or phosphate.

### e) Imatinib Prodrugs

In another aspect of the present invention pharmaceutical acceptable imatinib prodrug salts are provided such as alaninate, argininate, aspartate, glutamate, glycinate, histidinate, leucinate, prolinate, serinate, threoninate, tryptophanate, tyrosinate or cycteinate. It is within the scope of the present invention to use imatinib prodrugs for the preparation of suitable imatinib formulations for inhalative delivery by soft mist inhalers or modern nebulizers.

### 2) Nebulization of Imatinib Solutions

Another aspect of the present invention is the topical delivery of imatinib via the inhalative route in order to reduce side effects of the oral imatinib administration. In this regard, imatinib solutions are optimized for nebulization by different soft mist inhalers and nebulizers. Furthermore, soft mist inhalers and nebulizers are provided and characterized for the nebulization of highly concentrated imatinib solutions. Challenges include delivery of viscous drug solutions that can clog the apertures or pores and lead to inefficient or inaccurate inhalative drug delivery to patients or render the device inoperable. Addition or use of chaotropic compounds such as ethanol, urea, aldols, propylene glycol, ethylene glycol, polyethylene glycol or ectoin not only enhance the solubility and stability of aqueous imatinib solutions but also improve their capability of being nebulized by soft mist inhalers and nebulizers. The beneficial effects of addition or use of such compounds regarding the nebulization characteristics of imatinib solutions will become clear on the basis of the following examples.

### a) Soft Mist Inhalers

### Medspray™ Wet Aerosol Inhaler

The Medspray™ wet aerosol inhaler is a hand-held, preservative-free, non-pressurized metered dose device containing micro-engineered nozzles produced by wafer stepper lithography and etching techniques. The aerosol is produced according to the principle of Rayleigh break-up, with liquid being dispersed into droplets by pressing the drug solution through an array of nozzles with mechanical means. The drug solution can be stored in a container with a mechanical pump system or in pre-filled glass syringes with the soft mist nozzles already mounted. Different nozzles can be used to target a specific site in the respiratory tract. The mechanical energy for the aerosolization process is for example provided by a spring which is loaded and released by the patient.

### Example 5

In an in vitro nebulization study, the feasibility to nebulize different imatinib mesylate solutions by the Medspray™ wet aerosol inhaler was evaluated. A 1 ml syringe was filled with the formulation to be tested, a single nozzle unit of Medspray™ wet aerosol inhaler was then fixed on the syringe by a luer adapter. The nozzle pore size was 1.9 μm. The aerosol was produced by pressing the piston of the syringe manually, thereby forcing the solution through the nozzle pores. When using a. dest as solvent, imatinib mesylate solution with a maximum concentration of 210 mg/ml is capable of being nebulized and forming a soft mist. When using ethanol 10% (v/v) or glycerol 1% (v/v), solutions with a concentration of 240 mg/ml were nebulizable; a mixture of ethanol 10% (v/v) and glycerol 1% (v/v) further increased the maximum imatinib concentration of the solution to 250 mg/ml capable of being nebulized.

### Example 6

In a second in vitro nebulization study, different imatinib mesylate solutions with a fixed imatinib concentration of 200 mg/ml were investigated by a formulation acceptance test. Briefly, the imatinib solution was filled in a 1 ml syringe, a single nozzle unit (SNU) of Medspray™ wet aerosol inhaler was fixed at the tip of the syringe with a luer adapter. Syringe and SNU were then placed in a customized syringe pump equipped with pressure sensors assuring a continuous flow of the imatinib formulation through the SNU to determine the spray ability and to assess the dry spray pressure and relative viscosity compared to a. dest (table 1).

TABLE 1

| Imatinib | | Dry spray | |
|---|---|---|---|
| mesylate | spray | pressure | Relative |
| 200 mg/ml | ability | [bar] | viscosity |
| a. dest | Yes | 65 | 5.4 |
| glycerol 1% (v/v) | Yes | 62 | 5.5 |
| ethanol 5% (v/v) + | Yes | 57 | 5.3 |
| glycerol 1% (v/v) | | | |
| ethanol 10% (v/v) + | Yes | 60 | 5.3 |
| glycerol 1% (v/v) | | | |
| ethanol 10% (v/v) | Yes | 48 | 4.7 |

All tested solutions with an imatinib mesylate concentration of 200 mg/ml were capable of being nebulized forming a soft mist. Dry spray pressure was reduced by addition of glycerol and/or ethanol compared to a. dest, with ethanol 10% (v/v) as solvent displaying the lowest relative viscosity and the lowest dry spray pressure. The experiments demonstrate that the capability to be nebulized of highly concentrated imatinib mesylate aqueous solutions is increased when adding to a. dest chaotropic compounds such as ethanol, urea, aldols, propylene glycol, ethylene glycol, polyethylene glycol or ectoin, either alone or in different mixtures.

### Respimat™

The Respimat™ soft mist inhaler is a hand-held, pocket-sized device generating a single-breath, inhalable aerosol with slow velocity and long spray duration. By forcing non-pressurized drug solution through a two-channel nozzle (uniblock) using mechanical power, the solution is accelerated and split into two converging jets which collide at a certain angle, causing the drug solution to disintegrate into respirable droplets. The mechanical energy for the aerosolization process is provided by rotating the bottom of the device by 180° building up tension in a spring around the flexible drug container. When actuated by the patient, energy from the spring is released and imposes pressure on the flexible container holding the liquid drug formulation, whereby a metered-dose of liquid is forced through two nozzles and dispersed into an inhalable aerosol. The Respimat™ is already marketed, and for example available for the aerosol administration of tiotropium in COPD.

### Example 7

In an in vitro nebulization study, the feasibility of delivering imatinib mesylate solution by the Respimat™ was evaluated. The physical aerosol characteristics and the output of placebo Respimat™ and Respimat™ filled with different imatinib mesylate solutions were assessed. In order to compare particle size distribution of the two different solutions, the mass median aerodynamic diameters (MMAD) of the aerosol droplets were determined using laser light scattering (Sympatec™, Clausthal-Zellerfeld, Germany). The measurements (five runs of 1 sec duration, sampling rate 50 ms) were performed without additional air flow, with a distance between mouthpiece and laser beam of 5 cm. The data were analyzed in MIE mode, the density of the nebulized solution was set equal to unit density and thus the measured volume median diameter (VMD) equaled the mass median aerodynamic diameter. The fine particle fraction (FPF) was defined as the mass of particles <5.25 μm in size within the total emitted dose divided by the total emitted dose of aerosol particles. The geometric standard deviation (GSD) was calculated from the laser diffraction values according to the following equation:

$$G S D = \sqrt{\frac{84 \% undersize}{16 \% undersize}}$$

To assess the aerosol volume emitted by one puff from the Respimat™, the drug container of the device was weighed before and after a series of 20 consecutive puffs.

Firstly, the parameters were assessed for the placebo Respimat™. Following the experiments with the placebo Respimat™, the drug container was completely emptied by a syringe. After weighing the drug container, 2.0 ml of a solution containing ethanol 10% (v/v)+glycerol 1% (v/v) was filled in the drug container by a syringe. After priming the system with 10 puffs, the next series of experiments was performed to obtain the aerosol parameters. Following the experiments with ethanol 10% (v/v)+glycerol 1% (v/v), the drug container was completely emptied by a syringe. After weighing the drug container, 2.0 ml of a solution containing ethanol 10% (v/v), glycerol 1% (v/v) and imatinib mesylate 200 mg/ml was filled in the drug container by a syringe. After priming the system with 10 puffs, the next series of experiments was performed to obtain the aerosol parameters. This procedure was repeated for each tested imatinib solution, with firstly flushing the Respimat™ by the corresponding solvent. The results are summarized in the following table 2:

TABLE 2

| | | | | Volume |
|---|---|---|---|---|
| | MMAD | | FPF | per puff |
| Respimat ™ | (μm) | GSD | (%) | (μl) |
| Placebo | 5.04 ± 0.35 | 2.00 ± 0.14 | 53.3 ± 4.5 | 17.4 |
| ethanol 10% (v/v) + | 5.32 ± 0.30 | 2.04 ± 0.07 | 49.5 ± 3.2 | 16.9 |
| glycerol 1% (v/v) | | | | |
| in a. dest | | | | |
| ethanol 10% (v/v) + | 12.79 ± 0.61 | 1.55 ± 0.03 | 20.9 ± 1.0 | 16.0 |
| glycerol 1% (v/v) + | | | | |
| imatinib mesylate | | | | |
| 200 mg/ml | | | | |
| ethanol 10% (v/v) + | 6.64 ± 0.30 | 1.74 ± 0.08 | 45.7 ± 2.9 | 16.5 |
| glycerol 1% (v/v) + | | | | |
| imatinib mesylate | | | | |
| 175 mg/ml | | | | |
| imatinib mesylate | 5.47 ± 0.40 | 1.94 ± 0.10 | 51.7 ± 3.9 | 17.0 |
| 125 mg/ml | | | | |
| in a. dest | | | | |
| ethanol 10% (v/v) + | 12.94 ± 0.78 | 1.68 ± 0.05 | 20.4 ± 1.9 | 15.9 |
| imatinib mesylate | | | | |
| 225 mg/ml | | | | |
| ethanol 10% (v/v) + | 6.81 ± 0.78 | 1.80 ± 0.07 | 40.4 ± 3.7 | 16.2 |
| imatinib mesylate | | | | |
| 175 mg/ml | | | | |

MMAD: mass median aerodynamic diameter,
GSD: geometric standard deviation,
FPF: fine particle fraction, mean ± standard deviation, n = 5

### Other Soft Mist Inhalers

It is within the scope of the present invention to use soft mist inhaler other than Medspray™ wet aerosol inhaler or Respimat™ to administer highly concentrated imatinib solutions by inhalation to PH patients. For example, the soft mist inhaler may be the Pneuma Respiratory soft mist inhaler (Pneuma Respiratory Inc., Boone, N.C., USA) comprising a piezoelectric actuator and an aperture plate, the aperture plate having a plurality of openings and the piezoelectric actuator operable to oscillate the aperture plate at a frequency to thereby generate an ejected stream of droplets. In another embodiment, the soft mist inhaler is the Softhale soft mist inhaler (Softhale NV, Diepenbeek, Belgium) using a mechanically-driven impinging working principle to create a respirable aerosol.

### b) Vibrating Mesh Nebulizers

Vibrating mesh nebulizers are nebulizers based on the vibrating mesh technology to produce the aerosol from liquids. The technology comprises a plate perforated with precision formed holes vibrating app. 100,000 times per second, to produce the optimum particle size for deep lung penetration. Exemplary embodiments provided in accordance with the disclosed subject matter include, but are not limited to, the Pari eFlow™ Rapid and the Aeroneb™ Solo.

### Example 8 Nebulization of Imatinib Mesylate Solutions by Pari eFlow™ Rapid

In an in vitro nebulization study, the feasibility of delivering imatinib mesylate solutions by the Pari eFlow™ Rapid was evaluated. The physical aerosol characteristics and the output of the Pari eFlow™ Rapid filled with physiologic saline and different imatinib solutions were measured. To compare particle size distribution of the different solutions, the mass median aerodynamic diameters (MMAD) of the aerosol droplets were determined using laser light scattering (Sympatec™, Clausthal-Zellerfeld, Germany). The measurements (five runs of 1 sec duration, sampling rate 50 ms) were performed without additional air flow, with a distance between mouthpiece and laser beam of 5 cm. The data were analyzed in MIE mode, the density of the nebulized solution was set equal to unit density and thus the measured volume median diameter (VMD) equaled the mass median aerodynamic diameter. The fine particle fraction (FPF) was defined as the mass of particles <5.25 μm in size within the total emitted dose divided by the total emitted dose of aerosol particles. The geometric standard deviation (GSD) was calculated from the laser diffraction values according to the following equation:

$$G S D = \sqrt{\frac{84 \% undersize}{16 \% undersize}}$$

To assess the aerosol output, the nebulizer was weighed before and after a nebulization period of three minutes.

For the nebulization experiments, 2 ml of physiological saline or imatinib solution was filled in the medication chamber of the device. After an initial nebulization phase of 30 seconds the measurements were started.

TABLE 3

| | MMAD | | | Output |
|---|---|---|---|---|
| Pari eFlow ™ Rapid | (μm) | GSD | FPF (%) | (mg/min) |
| NaCl 0.9% | 5.41 ± 0.02 | 1.82 ± 0.03 | 47.8 ± 0.3 | 787 ± 10 |
| imatinib mesylate 200 mg/ml | 4.27 ± 0.20 | 1.70 ± 0.04 | 69.0 ± 4.7 | 110 ± 21 |
| in a. dest | | | | |
| ethanol 10% (v/v) + glycerol | 8.31 ± 0.19 | 1.66 ± 0.06 | 19.4 ± 2.9 | 309 ± 125 |
| 1% (v/v) in a. dest | | | | |
| imatinib mesylate 200 mg/ml | 3.92 ± 0.10 | 1.65 ± 0.02 | 74.8 ± 1.7 | 216 ± 28 |
| in ethanol 10% (v/v) | | | | |
| imatinib mesylate 200 mg/ml | 3.80 ± 0.17 | 1.55 ± 0.03 | 80.3 ± 4.3 | 193 ± 54 |
| in ethanol 10% (v/v) + glycerol | | | | |
| 1% (v/v) | | | | |
| imatinib mesylate 200 mg/ml | 3.53 ± 0.25 | 1.68 ± 0.19 | 64.3 ± 9.0 | 221 ± 35 |
| in ethanol 5% (v/v) + glycerol | | | | |
| 1% (v/v) | | | | |
| imatinib mesylate 100 mg/ml | 5.13 ± 0.22 | 1.78 ± 0.05 | 50.1 ± 2.8 | 547 ± 19 |
| in a. dest | | | | |
| imatinib mesylate 125 mg/ml | 4.81 ± 0.11 | 1.74 ± 0.03 | 65.2 ± 4.1 | 433 ± 38 |
| in ethanol 10% (v/v) | | | | |
| imatinib mesylate 125 mg/ml | 4.66 ± 0.14 | 1.67 ± 0.04 | 68.9 ± 4.0 | 411 ± 31 |
| in ethanol 10% (v/v) + glycerol | | | | |
| 1% (v/v) | | | | |

MMAD: mass median aerodynamic diameter,
GSD: geometric standard deviation,
FPF: fine particle fraction,
n.d.: not done, mean ± standard deviation, n = 5

Significant dripping of the solution through the vibrating plate was observed for imatinib mesylate 200 mg/ml in a. dest and for imatinib 200 mg/ml in ethanol 10% (v/v)+glycerol 1% (v/v).

### Example 9 Nebulization of Imatinib Mesylate Solutions by Aeroneb™ Solo

In an in vitro nebulization study, the feasibility of delivering imatinib mesylate solutions by the Aeroneb™ Solo was evaluated. The physical aerosol characteristics and the output of Aeroneb™ Solo filled with physiologic saline and the imatinib solution were measured. To compare particle size distribution of the different solutions, the mass median aerodynamic diameters (MMAD) of the aerosol droplets were determined using laser light scattering (Sympatec™, Clausthal-Zellerfeld, Germany). The measurements (five runs of 1 sec duration, sampling rate 50 ms) were performed without additional air flow, with a distance between mouthpiece and laser beam of 5 cm. The data were analyzed in MIE mode, the density of the nebulized solution was set equal to unit density and thus the measured volume median diameter (VMD) equaled the mass median aerodynamic diameter. The fine particle fraction (FPF) was defined as the mass of particles <5.25 μm in size within the total emitted dose divided by the total emitted dose of aerosol particles. The geometric standard deviation (GSD) was calculated from the laser diffraction values according to the following equation:

$$G S D = \sqrt{\frac{84 \% undersize}{16 \% undersize}}$$

To assess the aerosol output, the nebulizer was weighed before and after a nebulization period of three minutes.

For the nebulization experiments, 2 ml of physiological saline or imatinib solution was filled in the medication chamber of the device. After an initial nebulization phase of 30 seconds the measurements were started.

TABLE 4

| | MMAD | | | Output |
|---|---|---|---|---|
| Aeroneb ™ Solo | (μm) | GSD | FPF (%) | (mg/min) |
| NaCl 0.9% | 5.49 ± 0.37 | 2.01 ± 0.07 | 47.6 ± 4.2 | 651 ± 59 |
| Imatinib mesylate 225 mg/ml | 7.51 ± 5.66 | 1.95 ± 0.61 | 55.5 ± 23.1 | 102 ± 37 |
| in ethanol 10% (v/v) | | | | |
| imatinib mesylate 200 mg/ml | 3.89 ± 0.46 | 2.02 ± 0.09 | 67.3 ± 7.2 | 16 ± 4 |
| in a. dest | | | | |
| imatinib mesylate 100 mg/ml | 4.84 ± 0.38 | 2.00 ± 0.08 | 61.3 ± 6.6 | 411 ± 33 |
| in a. dest | | | | |
| imatinib mesylate 200 mg/ml | 4.27 ± 0.80 | 1.82 ± 0.20 | 65.8 ± 7.2 | 81 ± 21 |
| in ethanol 10% (v/v) + glycerol | | | | |
| 1% (v/v) | | | | |
| imatinib 200 mesylate mg/ml | 4.24 ± 0.75 | 2.03 ± 0.21 | 62.8 ± 6.5 | 64 ± 14 |
| in ethanol 5% (v/v) + Glycerol | | | | |
| 1% (v/v) | | | | |
| imatinib 125 mesylate mg/ml | 5.07 ± 0.41 | 1.99 ± 0.11 | 56.6 ± 5.3 | 365 ± 26 |
| in ethanol 10% (v/v) + glycerol | | | | |
| 1% (v/v) | | | | |

MMAD: mass median aerodynamic diameter,
GSD: geometric standard deviation,
FPF: fine particle fraction, mean ± standard deviation, n = 5
Significant dripping through the vibrating plate was observed for all tested imatinib containing solutions.

### c) Jet Nebulizers

Jet nebulizers are widely used in respiratory medicine to produce aerosols from liquids. The operation of a jet nebulizer requires a pressurized gas supply as the driving force for liquid atomization. Compressed gas is delivered through a jet, causing a region of negative pressure. The solution to be aerosolized is entrained into the gas stream and is sheared into a liquid film. This film is unstable and breaks into droplets.

### Example 10 Nebulization of Imatinib Mesylate Solutions by Pari LC Sprint™

In an in vitro nebulization study, the feasibility of delivering imatinib mesylate solutions by the jet-nebulizer Pari LC Sprint™ with Pari Boy compressor was evaluated. The physical aerosol characteristics and the output of the Pari LC Sprint™ filled with physiologic saline and different imatinib solutions were measured. To compare particle size distribution of the different solutions, the mass median aerodynamic diameters (MMAD) of the aerosol droplets were determined using laser light scattering (Sympatec™, Clausthal-Zellerfeld, Germany). The measurements (five runs of 1 sec duration, sampling rate 50 ms) were performed without additional air flow, with a distance between mouthpiece and laser beam of 5 cm. The data were analyzed in MIE mode, the density of the nebulized solution was set equal to unit density and thus the measured volume median diameter (VMD) equaled the mass median aerodynamic diameter. The fine particle fraction (FPF) was defined as the mass of particles <5.25 μm in size within the total emitted dose divided by the total emitted dose of aerosol particles. The geometric standard deviation (GSD) was calculated from the laser diffraction values according to the following equation:

$$G S D = \sqrt{\frac{84 \% undersize}{16 \% undersize}}$$

To assess the aerosol output, the nebulizer was weighed before and after a nebulization period of three minutes volume.

For the nebulization experiments, 4 ml of physiological saline or imatinib solution was filled in the medication chamber of the device. After an initial nebulization phase of 30 seconds the measurements were started.

TABLE 5

| | MMAD | | FPF | Output |
|---|---|---|---|---|
| Pari LC Sprint ™ | (μm) | GSD | (%) | (mg/min) |
| NaCl 0.9% | 3.60 ± 0.11 | 1.80 ± 0.09 | 70.4 ± 3.3 | 597 ± 16 |
| imatinib | 1.74 ± 0.06 | 1.61 ± 0.04 | 79.7 ± 3.0 | 158 ± 17 |
| mesylate 250 | | | | |
| mg/ml in a. dest | | | | |
| imatinib | 2.73 ± 0.08 | 1.69 ± 0.07 | 72.1 ± 2.8 | 294 ± 25 |
| mesylate 250 | | | | |
| mg/ml in | | | | |
| ethanol 10% (v/v) | | | | |

In summary, nebulization of imatinib solutions by vibrating mesh nebulizers or jet nebulizers is feasible. The output of vibrating mesh or jet nebulizers is, however, significantly reduced when nebulizing imatinib mesylate solutions with imatinib concentrations of more than 100 mg/ml, irrespective of the specific composition of the solvent.

### 3) Use of Imatinib as Aerosol Therapy to Treat Pulmonary Hypertension

Oral imatinib mesylate has already shown to be effective in PAH, significantly improving 6-minute walk distance as primary outcome in a randomized, double-blind, placebo-controlled 24-week trial in 202 PAH-patients (Hoeper et al., Circulation 2013 127(10):1128-38). The documented side effect profile of oral imatinib, however, prevented the further use and approval of this drug for treatment of pulmonary hypertension.

In order to avoid systemic side effects of oral imatinib and to facilitate its use in pulmonary hypertension, the present invention provides methods, compositions and kits for administering imatinib solutions as aerosol to treat pulmonary hypertension.

Patients treated with the methods, compositions and kits disclosed herein suffer from pulmonary hypertension or from other disorders of the pulmonary vasculature or pulmonary circulation. For example, the subjects may belong to one of the following five groups of pulmonary hypertension according to the WHO: Group 1, Pulmonary arterial hypertension (PAH) including subclasses 1.1 Idiopathic PAH, 1.2 Heritable PAH, 1.3 Drug- and toxin-induced PAH, 1.4 PAH associated with 1.4.1 Connective tissue disease, 1.4.2 HIV infection, 1.4.3 Portal hypertension, 1.4.4 Congenital heart disease, 1.4.5 Schistosomiasis, 1.5 PAH long-term responders to calcium channel blockers; 1.6 PAH with overt features of venous/capillaries (PVOD/PCH) involvement and 1.7 Persistent PH of the newborn syndrome; Group 2, Pulmonary hypertension due to left heart disease including subclasses 2.1 PH due to heart failure with preserved LVEF, 2.2 PH due to heart failure with reduced LVEF, 2.3 Valvular heart disease and 2.4 Congenital/acquired cardiovascular conditions leading to post-capillary PH; Group 3, Pulmonary hypertension due to lung disease and/or hypoxia including subclasses 3.1 Obstructive lung disease 3.2 Restrictive lung disease, 3.3 Other lung disease with mixed restrictive/obstructive pattern, 3.4 Hypoxia without lung disease and 3.5 Developmental lung disorders; Group 4, Pulmonary hypertension due to pulmonary artery obstructions including subclasses 4.1 Chronic thromboembolic PH and 4.2 Other pulmonary artery obstructions; Group 5, Pulmonary hypertension with unclear and/or multifactorial mechanisms including subclasses 5.1 Hematological disorders 5.2 Systemic and metabolic disorders, 5.3 Others and 5.4 Complex congenital heart disease disorder. The subject may belong to functional class I, class II, class III or class IV according to the functional classification of pulmonary hypertension of the World Health Organization, modified after the New York Heart Association functional classification.

The subject may have no medication, or receive supportive therapy such as oral anticoagulants, diuretics, oxygen, digoxin. In addition, therapy may include high-dose calcium channel blockers or specific drugs approved for PH, encompassing endothelin receptor antagonists such as ambrisentan (oral), bosentan (oral) or macitentan (oral), phosphodiesterase type 5 inhibitors and guanylate cyclase stimulators or activators such as sildenafil (oral, intravenous), tadalafil (oral), vardenafil (oral) or riociguat (oral), prostacyclin analogues and prostacyclin receptor agonists such as beraprost (oral), epoprostenol (intravenous), iloprost (aerosol, intravenous), treprostinil (aerosol, subcutaneous, intravenous, oral) or selexipag (oral). These mainly vasodilatory drugs may be administered to the subject as monotherapy or as combination therapy using two or more drugs simultaneously.

Also included in the present invention is the use of further future PH-specific drugs, with such drugs mainly focusing on typical characteristics of pulmonary vascular remodeling.

The imatinib inhalation disclosed herein may be administered to therapy-naïve patients or to patients on supportive therapy. In addition, imatinib aerosol therapy be in addition to chronic therapy using one or more PH-specific drugs.

### Dosing

According to the preexisting studies, a single dose of 400 mg imatinib mesylate per day seems to represent the optimal and required dose to treat pulmonary hypertension. In consideration of the dose-saving aspect of an inhalative approach, a significant lower inhaled dose of approximately one half to one-twentieth of the oral 400 mg imatinib can be projected to achieve the full efficacy profile of the drug, thereby minimizing systemic side effects.

It is within the present invention to provide daily doses of 5 to 400 mg imatinib delivered via the inhalative route and deposited in the respiratory tract (total lung dose). In a preferred embodiment the daily total lung dose of inhaled imatinib is 20 mg to 250 mg, or 25 mg to 150 mg, or 50 mg to 100 mg. The dose can preferably be administered once daily. Alternatively, the daily dose can be split and inhaled twice daily.

The following is a non-exhaustive list of possible combinations of imatinib solutions with different drug concentrations and output of the inhalers or nebulizers to deliver the claimed dose for imatinib aerosol therapy of pulmonary hypertension.

### Example 11 Imatinib Aerosol Therapy with Medspray™ Wet Aerosol Inhaler

Table 6 depicts the daily total lung dose of imatinib depending on the emitted aerosol volume, number of puffs, imatinib concentration and daily treatment sessions when inhaling by the Medspray™ wet aerosol inhaler.

TABLE 6

| | | | | Daily |
|---|---|---|---|---|
| | | | Number of | total lung |
| | Imatinib | Volume | puffs per | dose of |
| | concentration | per puff | treatment | imatinib |
| Medspray ™ | (mg/ml) | (μl) | session | (mg) |
| ethanol | 200 | 50 | 5 | 35 |
| 10% (v/v) | | | | |
| ethanol | 200 | 50 | 10 twice | 70 |
| 10% (v/v) | | | daily | |
| ethanol | 200 | 50 | 10 | 70 |
| 10% (v/v) | | | | |
| ethanol | 200 | 50 | 10 twice | 140 |
| 10% (v/v) | | | daily | |

The deposition fraction, i.e. the fraction of inhaled aerosol that deposits in the respiratory tract, was assumed to be 0.7 for the Medspray™ wet aerosol inhaler.

### Example 12 Imatinib Aerosol Therapy with Respimat™

Table 7 depicts the daily total lung dose of imatinib depending on the emitted aerosol volume, number of puffs, imatinib concentration and daily treatment sessions when inhaling by the soft mist inhaler Respimat™.

TABLE 7

| | | | | Daily |
|---|---|---|---|---|
| | | | Number of | total lung |
| | Imatinib | Volume | puffs per | dose of |
| | concentration | per puff | treatment | imatinib |
| Respimat ™ | (mg/ml) | (μl) | session | (mg) |
| ethanol | 175 | 20 | 10 | 24.5 |
| 10% (v/v) | | | | |
| ethanol | 175 | 20 | 10 twice daily | 49 |
| 10% (v/v) | | | | |

The deposition fraction, i.e. the fraction of inhaled aerosol that deposits in the respiratory tract, was assumed to be 0.7 for the Medspray™ wet aerosol inhaler.

### Example 13 Imatinib Aerosol Therapy with Pari eFLOW™ Rapid

Table 8 depicts the daily total lung dose of imatinib depending on the emitted aerosol volume, imatinib concentration and daily treatment sessions when inhaling by the vibrating mesh nebulizer Pari eFLOW™ Rapid.

TABLE 8

| | | | | Daily |
|---|---|---|---|---|
| | | | | total lung |
| Pari | Imatinib | | Duration of | dose of |
| eFLOW ™ | concentration | Output | inhalation | imatinib |
| Rapid | (mg/ml) | (μl/min) | (min) | (mg) |
| ethanol | 125 | 400 | 1 | 25 |
| 10% (v/v) | | | | |
| ethanol | 125 | 400 | 2, 3, 4, | 50, 75, |
| 10% (v/v) | | | 5, 10 | 100, 250 |
| ethanol | 125 | 400 | 8 | 200 |
| 10% (v/v) | | | | |
| ethanol | 125 | 400 | 8 min | 400 |
| 10% (v/v) | | | twice daily | |

The deposition fraction, i.e. the fraction of inhaled aerosol that deposits in the respiratory tract, was assumed to be 0.5 for the Pari eFLOW™ nebulizer.

### Example 14 Imatinib Aerosol Therapy with Pari LC Sprint™

Table 9 depicts the daily total lung dose of imatinib depending on the emitted aerosol volume and number of puffs, imatinib concentration and daily treatment sessions when inhaling by the soft mist inhaler Pari LC Sprint™

TABLE 9

| | | | | Daily |
|---|---|---|---|---|
| | | | | total lung |
| | Imatinib | | Duration of | dose of |
| Pari LC | concentration | Output | inhalation | imatinib |
| Sprint ™ | (mg/ml) | (μl/min) | (min) | (mg) |
| ethanol | 250 | 250 | 1 | 25 |
| 10% (v/v) | | | | |
| ethanol | 250 | 250 | 2, 3, 4, | 50, 75, |
| 10% (v/v) | | | 5, 10 | 100, 250 |

The deposition fraction, i.e. the fraction of inhaled aerosol that deposits in the respiratory tract, was assumed to be 0.4 for the Pari LC Sprint™ jet nebulizer.

The use of portable, prefilled soft most inhalers for aerosol delivery of imatinib is preferred, avoiding multiple steps necessary to prepare and perform inhalation maneuvers when using vibrating mesh nebulizers or jet nebulizers: opening of an ampoule containing imatinib, assembly of the nebulizer, transfer of the imatinib solution into the nebulization chamber of the nebulizer by a pipette or syringe, inhalation with a duration of several minutes, removal of the residual solution in the nebulization chamber, cleaning of the different pieces of the nebulizer.

1. A method of treating a patient suffering from pulmonary hypertension, comprising:
(a) providing stable and highly concentrated imatinib solutions;
(b) providing an aerosol producing device capable of aerosolizing such stable and highly concentrated imatinib solutions;
(c) administering to the patient an effective dose of imatinib by inhalation.

2. A method of claim 1, wherein imatinib is a pharmaceutical acceptable salt of imatinib, with the salt being mesylate, tartrate, citrate, maleate, fumarate, succinate, benzoate, besylate, tosylate, palmoate, formate, malonate, napsylate, salysilate, cyclohexane sulfamate, lactate, mandelate, glutarate, adipate, squarate, vallinate, oxaloacetate, ascorbate and sulfate salts, oxalate, p-toluene sulfonate, naphthalene sulfonate, benzene sulfonate, nitrate, phosphate, acetate, lysinate, lysinate-HCL, or arginate. Preferably, the imatinib salt is selected from the group of highly water-soluble salts including mesylate, maleate, tartrate, malonate, succinate, tosylate, oxalate or phosphate. In a preferred embodiment the imatinib salt is imatinib mesylate.

3. A method of claim 1, wherein imatinib is a pharmaceutical acceptable imatinib prodrug salt such as alaninate, argininate, aspartate, glutamate, glycinate, histidinate, leucinate, prolinate, serinate, threoninate, tryptophanate, tyrosinate or cycteinate.

4. A method of claim 1, wherein the solvent for imatinib, imatinib salts or imatinib prodrug salts is aqua destillata.

5. A method of claim 1, wherein the solvent for imatinib, imatinib salts or imatinib prodrug salts is ethanol, or glycerol, or propylene glycol, or ethylene glycol, or polyethylene glycol, or mixtures thereof.

6. A method of claim 1, wherein chaotropic compounds such as ethanol, urea, aldols, propylene glycol, ethylene glycol, polyethylene glycol or ectoin, alone or in combination, are added to aqua destillata for preparing an aqueous solution of imatinib, imatinib salts or imatinib prodrug salts. In preferred embodiments, ethanol 10% (v/v), or ethanol 10% (v/v) and glycerol 1% (v/v) are used as solvents.

7. A method of claim 1, wherein the concentration of imatinib in the imatinib solutions ranges from 50 to 500 mg/ml, preferably from 100 to 250 mg/ml.

8. A method of claim 1, wherein the aerosol producing device is selected from the group of soft most inhalers, vibrating mesh nebulizers or jet nebulizers, preferably from the group of portable, prefilled soft mist inhalers. In preferred embodiments, the soft mist inhaler is the Medspray™ wet aerosol inhaler or the Respimat™.

9. A method of claim 1, wherein the effective dose of imatinib delivered to a patient ranges from 5 to 400 mg per day, administered via inhalation and deposited in the respiratory tract (i.e. total lung dose). In preferred embodiments the daily total lung dose of inhaled imatinib is 20 mg to 250 mg, or 25 mg to 150 mg, or 50 mg to 100 mg.

10. A method of claim 1, wherein the effective dose of imatinib is administered once daily. In a further embodiment, the daily dose can be split and inhaled twice daily.

11. A method of claim 1, wherein the patient suffering from pulmonary hypertension is
(a) therapy-naïve,
(b) on supportive therapy and/or
(c) receiving chronic therapy with one or more approved PH-specific drugs select-ed from the group of endothelin receptor antagonists (e.g. ambrisentan, bosentan, macitentan), of phosphodiesterase type 5 inhibitors and guanylate cyclase stimulators (e.g. sildenafil, tadalafil, vardenafil, or riociguat), of prostacyclin analogues and prostacyclin receptor agonists (e.g. beraprost, epoprostenol, iloprost, trepros-tinil, or selexipag), and/or
(d) receiving disease-modifying drugs addressing vascular remodeling, alone or in combination with therapy according to (b) or (c).
